(19) United States
(12) Patent Application Publication
(10) Pub. No.: US 2003/0217370 A1
(43) Pub. Date: Nov. 20, 2003

(54) Transgenic animal expressing alpha-synuclein and uses thereof

(76) Inventors: Benoit I. Giasson, Lansdowne, PA (US); John Q. Trojanowski, Philadelphia, PA (US); Virginia M.-Y. Lee, Philadelphia, PA (US)

Correspondence Address: Licata & Tyrrell P.C., 66 E. Main Street, Marlton, NJ, 08053, US

(21) Appl. No.: 10/150,043

(22) Filed: May 16, 2002

(51) Int. Cl. A01K 67/00

(52) U.S. Cl. 800/3; 800/8

(57) ABSTRACT

The present invention relates to transgenic animals expressing A53T α-Syn protein which are models for neurodegenerative disease progression. These animals may be used for screening candidate drug compounds effective against neurodegenerative diseases and other diseases involving α-synucleinopathy.

### BACKGROUND OF THE INVENTION

α-Synuclein (α-Syn) is a small (i.e. 140 amino acids) protein predominantly expressed in neurons and concentrated at synaptic terminals (George, et al. (1995) Neuron 15:361-372; Jakes, et al. (1994) FEBS Lett. 345:27-32). Although the function(s) of α-Syn is ill-defined, evidence suggests roles in neural plasticity and regulation of synaptic vesicle pools (George, et al. (1995) Neuron 15:361-372; Murphy, et al. (2000) J. Neurosci. 20:3214-3220). α-Syn is the major component of pathological intracellular proteinaceous inclusions characteristic of specific neurological disorders including Parkinson's disease (PD), Lewy body (LB) variant of Alzheimer's disease (LBVAD), dementia with LB (DLB), neurodegeneration with brain iron accumulation type-1 (NBIA-1) (formerly known as Hallervorden-Spatz disease) and multiple system atrophy (MSA) (Baba, et al. (1998) Am. J. Pathol. 152:879-884; Duda, et al. (2000) J. Neuropathol. Exp. Neurol. 59:830-841; Galvin, et al. (2000) Am. J. Pathol. 157:361-368; Spillantini, et al. (1997) Nature 388:839-840; Spillantini, et al. (1998) Proc. Natl. Acad. Sci. U.S.A. 95:6469-6473; Tu, et al. (1998) Ann. Neurol. 44:415-422). The role of α-Syn pathology in a large number of neurodegenerative diseases is due to a dominantly inherited α-Syn substitution, A53T, found in at least 12 families with familial PD (Polymeropoulos, et al. (1997) Science 276:2045-2047; Golbe (1999) Mov Disord. 14:6-9; Spira, et al. (2001) Ann. Neurol. 49:313-319).

Neuronal α-Syn inclusions occur as classical LBs, i.e. round filamentous aggregates comprised of a core and halo detected by conventional histological stains especially in dopaminergic neurons of the substantia nigra pars compacta (SNpc) of PD patients (Formo (1996) J. Neuropathol. Exp. Neurol. 55:259-272). However, the vast majority of neuronal α-Syn lesions, including cortical LBs, other neuronal inclusions, neuroaxonal spheroids and dystrophic neurites, termed Lewy neurites, were not fully appreciated until recently, and are reliably detected by immunocytochemistry with antibodies to α-Syn (Baba, et al. (1998) Am. J. Pathol. 152:879-884; Spillantini, et al. (1997) Nature 388:839-840; Spillantini, et al. (1998) Proc. Natl. Acad. Sci. U.S.A. 95:6469-6473). Antibodies to different modified forms of α-Syn have been used to redefine the neuropathology of α-synucleinopathies. For example, in the Contursi kindred with the A53T α-Syn mutation, extensive α-Syn neuritic pathology was detected throughout the brain including limbic system, striatum, and locus coeruleus. Neuritic pathology is much more abundant than perikaryal inclusions and substantia nigra was not the most severely affected brain nuclei (Spira, et al. (2001) Ann. Neurol. 49:313-319). α-Syn inclusions also accumulate in oligodendrocytes of patients afflicted by MSA, but these lesions, termed glial cytoplasmic inclusions, are largely restricted to this disorder (Duda, et al. (2000) J. Neuropathol. Exp. Neurol. 59:830-841; Tu, et al. (1998) Ann. Neurol. 44:415-422). Finally, in situ and in vitro biochemical assessments indicate that all α-Syn inclusions consist of bundles of 10-25 nm filaments comprised of polymerized α-Syn (Baba, et al. (1998) Am. J. Pathol. 152:879-884; Giasson, et al. (1999) J. Biol. Chem. 274:7619-7622; Spillantini, et al. (1998) Proc. Natl. Acad. Sci. U.S.A. 95:6469-6473; Tu, et al. (1998) Ann. Neurol. 44:415-422).

PD is the most widely recognized α-synucleinopathy, since it is the most common movement disorder with a prevalence of 1% at 65 years of age and increasing to 4-5% by the age of 85 (de Rijk, et al. (1997) J. Neurol. Neurosurg. Psychiatry 62:10-15). It is characterized clinically by bradykinesia, resting tremor, rigidity, postural instability and periods of freezing (Simuni and Hurtig (2000) Parkinson's disease: the clinical picture. In: Neurodegenerative dementias, C. M. Clark and J. Q. Trojanoswki, eds. (New York: McGraw-Hill), pp. 193-203). In PD, neuronal α-Syn pathology is concentrated in brain stem nuclei, including the SNpc and locus coeruleus (Formo (1996) J. Neuropathol. Exp. Neurol. 55:259-272). On the other hand, DLB and LBVAD are characterized by dementia, parkinsonism, hallucinations, and the presence of widespread and abundant neuronal α-Syn inclusions and neurites (McKeith, et al. (1996) Neurology 47:1113-1124). Although α-Syn pathologies are the defining neuropathological hallmarks of α-synucleinopathies, their role in disease pathogenesis and how they contribute to impaired cellular function as well as brain degeneration remains unresolved. This is further confounded by the fact that a threonine at amino acid residue 53 is the normal α-Syn sequence found in rodent (Hsu, et al. (1998) J. Neurochem. 71:338-344). The observation that α-Syn polymerization in vitro is concentration-dependent (Giasson, et al. (1999) J. Biol. Chem. 274:7619-7622; Wood et al. (1999) J. Biol. Chem. 274:19509-19512) raises the possibility that an increased abundance of α-Syn may contribute to the formation of pathological inclusions.

Several transgenic mouse models expressing wild-type and mutant α-Syn have been described, but these models do not reveal differences between wild-type and mutant α-Syn nor do they fully relate the characteristics of human α-Syn pathology (Kahle, et al. (2000) J. Neurosci. 20:6365-6373; van der Putten, et al. (2000) J. Neurosci. 20:6021-6029). The comparison of these different models is complicated by the use of different promoters, which have varied expression in specific neuronal populations. The human platelet-derived growth factor-β (PDGF-β) and the murine tyrosine hydroxylase (Thy-1) promoter have been used to express human α-Syn in mice. When the PDGF-β promoter was used, expression of wild-type protein resulted in the formation of amorphous, non-filamentous α-Syn aggregates associated with impairment of motor function and reduction in striatal TH-terminals (Masliah, et al. (2001) Proc. Natl. Acad. Sci. U.S.A. 98:12245-12250). A subset of α-Syn inclusions also contained ubiquitin, which is characteristic of authentic human inclusions (Love and Nicoll (1992) Neuropathol. Appl. Neurobiol. 18:585-592).

Expression of wild-type and A53T or A30P mutant α-Syn in transgenic mice driven by the Thy-1 promoter results in the appearance of perikaryal and neuritic accumulations, without notable differences between wild-type and mutant proteins (Kahle, et al. (2000) J. Neurosci. 20:6365-6373; van der Putten, et al. (2000) J. Neurosci. 20:6021-6029). Mice expressing wild-type or A53T mutant α-Syn developed an early onset motor impairment (>3 weeks of age) as measured by rotating rod performance, and this phenotype was associated with axonal degeneration in the spinal root and muscle denervation (van der Putten, et al. (2000) J. Neurosci. 20:6021-6029). A subset of α-Syn inclusions in these mice were argyrophillic and immunoreactive for ubiquitin, but lacked the filamentous characteristic of authentic α-Syn inclusions. On the other hand, expression of wild-type or mutant α-Syn human α-Syn in Drosophilae results in the formation of filamentous α-Syn inclusions concomitant with the demise of dorsomedial dopaminergic neurons and impairment of locomotor function (Feany and Bender (2000) Nature 404:394-398). Furthermore, transgenic mice expression the A53T mutant α-Syn driven by a prion promoter have been described, but no phenotype was provided as these animals were being intercrossed to establish a +/−α-Syn background (Orrison, et al. (1999) American Society of Human Genetics 1999 Meeting, abstract #2746).

An animal model that displays a dramatic behavioral phenotype to reveal differences between wild-type and mutant α-Syn which fully relates the characteristics of human pathology where neuronal dysfunction coincides with the formation of α-Syn inclusions is needed. The present invention meets this long felt need.

### SUMMARY OF THE INVENTION

The invention provides transgenic animals expressing wild-type or mutant isoforms of human α-synuclein protein. The transgenic animals of the invention can be used for screening candidate drug compounds, which may be effective against neurodegenerative diseases and other diseases involving α-synucleinopathy. The transgenic animals of the invention can also be used in genetic crosses with other transgenic animals to model the progression of neurodegenerative disease.

### DETAILED DESCRIPTION OF THE INVENTION

The present invention relates to the generation and use of a transgenic animal that expresses wild-type or A53T mutant forms of α-synuclein. The immunological, histological, biochemical and ultrastructural properties of inclusions in such an animal closely resemble authentic human pathological inclusions. α-Syn inclusions of these models share many of the characteristic immunological properties of authentic human lesions.

Transgenic animals contain within their genetic material some genetic information from another organism. For example, a transgenic mouse might have a human gene inserted into its genetic material. Such foreign genetic material is referred to as a transgene.

Transgenic mice can be generated in a number of manners known to those of skill in the art. Typically, a transgenic animal is generated according to the following basic steps: producing a nucleic acid construct with a promoter and cDNA or gene sequences of interest; linearizing and injecting the transgene construct into the pronuclei of embryos; implanting the embryos into the uterus of a pseudo-pregnant female; screening the offspring that are born, i.e. live pups in the case of mice, for the presence of the transgene; and breeding the transgene-positive pups (founders) to obtain transgenic lines. Examples of these and related techniques are found in, e.g., Hogan, B., Costantini, F. & Lacy, E., eds., Manipulating the Mouse Embryo: A Laboratory Manual, Cold Spring Harbor Laboratory Press, Cold Spring Harbor, N.Y. (1986).

To generate the transgenic (Tg) mice described herein that express wild-type and mutant A53T human α-Syn, the respective cDNAs were cloned into the MoPrP.Xho expression plasmid which drives high expression of the transgene in most central nervous system neurons from a mouse prion promoter (Borchelt, et al. (1996) Genet. Anal. 13:159-163). Tg lines expressing wild-type (lines M7, M12 and M20) or mutant A53T (lines M83 and M91) human α-Syn were bred to homozygosity. The levels of α-Syn expression in homozygous Tg mice were determined by quantitative western blot (Table 1).

TABLE 1

| Tg Lines | Cortex | Spinal Cord |
|---|---|---|
| M7 | 3.8 ± 0.4 | 19.4 ± 3.2 |
| M12 | 2.6 ± 0.6 | 12.8 ± 3.5 |
| M83 | 4.6 ± 0.8 | 28.2 ± 8.8 |
| M91 | 4.8 ± 0.8 | 23.2 ± 3.8 |

Neuronal-specific enolase (NSE) levels were measured and used as a control for equivalent loading of protein extracts. The anti-α-Syn-specific antibody SNL-1, which reacts equally with murine and human α-Syn (Giasson, et al. (2000) J. Neurosci. Res. 59:528-533), was used to detect and quantify total α-Syn. Syn 208 antibody, which is specific for human α-Syn (Giasson, et al. (2000) J. Neurosci. Res. 59:528-533), was used to detect transgene expression exclusively. The relative levels of over-expression of α-Syn compared to endogenous levels were much higher in the spinal cord than in the cortex, but this is due to the lower amounts of endogenous α-Syn in the spinal cord than in the cerebral cortex. The absolute level of expression of α-Syn within the cortex, spinal cord, and cerebellum of each Tg line was similar. For example, the ratio of total α-Syn in the cortex versus cerebellum and spinal cord was 1.0±0.4:1.4±0.4:1.0±0.3 for Tg line M7 and 1.0±0.2:1.7±0.4:1.4±0.1 for Tg line M83. Homozygous mice lines M7, M83 and M91 expressed similar levels of α-Syn, while expression in line M12 was slightly lower in all three regions analyzed biochemically. Homozygous mice from line M20 expressed the highest levels of α-Syn than any other Tg mouse lines. The level of overexpression in hemizygous Tg mice was more than half the level of homozygotes. For example, the level of overexpression in the cortex of M20 hemizygotes was 5.6±0.7 compared to 6.9±0.7 for M20 homozygotes, while it was 3.3+0.5 for M83 hemizygotes compared to 4.6±0.8 for M83 homozygotes.

Homozygous Tg mice expressing wild-type or A53T mutant human α-Syn remained healthy without any overt phenotype up to the age of 7 months. At this point, mice did not have muscle weakness as determined by their ability to stand on a slanted surface. Furthermore, up to this age, homozygous mice expressing either wild-type (line M7) or A53T (line M83) human α-Syn did not exhibit any impaired performance on the rotarod task: nTg=306.2+11.0 (n=12); M7 homozygous=316.4+12.0 (n=12); M83 homozygous=349.3+17.3 (n=8). By 8 months of age, a few homozygous mice expressing A53T α-Syn began to develop a dramatic motor phenotype. The initial changes included neglect of grooming, weight loss and reduced ambulation. These changes were followed by severe movement impairment with resistance to passive movement and partial paralysis of limbs, accompanied by periods (several seconds) of freezing of a hind limb. Tremulous motion was observed in some recumbent animals possibly related to attempted muscular activity. Paralysis of the extremities usually began at a hind leg, but within a few days all four limbs became affected. Concurrently, mice were also unable to right themselves when placed on their sides and they developed hunched backs. The animals eventually were unable to stand up and support their own body weight. Affected mice became unable to feed themselves in a standard cage, but their life-span could be prolonged for a few days by bottom feeding. Within 10-21 days of the first signs of disease, animals were sacrificed to prevent suffering. Notably, by contrast to many other mouse models of human disease (Cote, et al. (1993) Cell 73:35-46; Ishihara, et al. (1999) Neuron 24:751-762; Lee, et al. (1994) Neuron 13:975-988), normal and affected α-Syn Tg mice did not show retraction of their hind limbs when held inverted by the tail.

All homozygous Tg animals from lines M83 and M91 developed the phenotype described above within 16 months of age. Many hemizygous animals from line M83 also developed the same phenotype, but the age of onset was between 22 and 28 months of age. None of the hemizygous mice from line M91 up to the age of 28 months were affected. No neurological phenotype was observed in any of the hemizygous or homozygous Tg mice expressing wild-type human α-Syn (lines M7 and M12) up to 28 months of age. Similarly, hemizygous and homozygous mice from line M20 as old as 24 months and 14 months, respectively, did not display any neurological phenotype.

To characterize the α-Syn pathology of mice expressing A53T human α-Syn, histological analysis was conducted. Immunostaining for α-Syn in homozygous mice of lines M7 and M12 up to the age of 26 months revealed the normal neuropil staining pattern expected for this protein (Giasson, et al. (2001) Exp. Neurol. 172:354-362; Jakes, et al. (1994) FEBS Lett. 345:27-32). Similar results were obtained for 24 month old hemizygous and 14 month old homozygous mice of line M20. The same immunostaining pattern was also observed in all homozygous Tg mice expressing the A53T mutant protein until the age of 6 months. Furthermore, no differences were detected in the localization of human or mouse α-Syn using species-specific antibodies. However, in homozygous A53T mutant α-Syn animals ranging between 8 to 16 months of age, α-Syn inclusions in the somatodendritic compartment and dystrophic neurites became abundant and widely distributed throughout the neuraxis. This finding is consistent with the A53T α-Syn mutation in humans, where inclusions are significantly more widespread throughout the neuraxis than in PD (Spira, et al. (2001) Ann. Neurol. 49:313-319). A high density of inclusions was observed in the spinal cord, throughout the brainstem, the deep cerebellar nuclei, deep cerebellar white matter, and some regions of the thalamus, such as the medioventral, ventromedial and paracentral nuclei. Similar to human disease (Shoji, et al. (2000) J. Neurol. Neurosurg. Psychiatry 68:605-608), a gradient of neuritic pathology was found in the striatum with the densest accumulation in the dorsolateral part. Moreover, sparse pathology was found in motor cortex. Other regions of the cerebral cortex, the olfactory bulb, and the hippocampus were devoid of α-Syn pathology. Certain cell populations were completely spared including the Purkinje cells and granular cells in the cerebellum and tyrosine hydroxylase (TH)-positive neurons of the substantia nigra. The pathology in TH expressing neurons is different from human disorders. Experiments using the TH promoter to express wild-type and mutant α-Syn selectively in these neurons did not result in the formation of inclusions (Matsuoka, et al. (2001) Neurobiol. Dis. 8:535-539).

In homozygous M83 and M91 A53T mutant α-Syn Tg mice, the earliest age that pathology was observed was 7 months of age with scant α-Syn inclusions detected. The same profile of α-Syn inclusions was only detected in hemizygous Tg mice of line M83 when animals between 22 and 28 months of age when animals developed the motor phenotype described above.

While many perikaryal α-Syn lesions filled the entire somatodendritic compartment, some were more distinct structures reminiscent of cortical LBs in humans. These LB-like inclusions were abundant in the raphe nuclei, the pons and less abundant in locus coeruleus. Some of the inclusions in the locus coeruleus were in TH-positive neurons. α-Syn inclusions in neuronal processes appeared as dystrophic neurites reminiscent of LNs in human diseases as well as larger neuroaxonal spheroids, characteristic of NBIA-1. Occasional α-Syn ovoids were also detected in the sciatic nerve.

α-Syn inclusions were detected with several antibodies (i.e. Syn 303, Syn 505, Syn 506 and Syn 514) raised to oxidized α-Syn that preferentially recognize pathological α-Syn in human brain tissue from patients with synucleinopathies. The labeling of inclusions with a panel of conventional α-Syn antibodies was variable: robust staining with SNL-4, moderate staining with Syn 202 and LB509, and weak staining with Syn 211, Syn 102, and Syn 204. These α-Syn lesions, especially those in neuronal processes, are strikingly reminiscent of inclusions found in human synucleinopathies including patients with the A53T substitution, and they do not contain α-Syn or y-Syn since they were not detected by anti-α-Syn (Syn 207) or anti-y-Syn (gamma-1) antibodies. Likewise, there was also a paucity of staining with phosphorylation-dependent anti-neurofilament (NF) antibodies (e.g. RMO55 and RMO24), but in contrast, another phosphorylation-dependent anti-NF antibody, RMO32, which was found previously to label many LBs in human PD and dementia with LB (DLB) cases (Schmidt, et al. (1991) Am. J. Pathol. 139:53-65), labeled some of the α-Syn inclusions. Approximately 10-25% of α-Syn inclusions also demonstrated ubiquitin immunoreactivity.

Consistent with neuronal injury in affected Tg mice expressing A53T α-Syn, significant astrocytic gliosis was observed with glial fibrillary acidic protein (GFAP) staining. However, quantitative analysis of motor spinal neurons did not reveal significant loss of these cells even in mice displaying severe motor impairment: motor impaired M83 Tg mouse=16.5±0.5 neurons/section and non-Tg mouse=16.7+0.8 neurons/section. This finding is consistent with the short time interval between appearance of pathological changes and presentation of phenotype changes. Similar to authentic human pathological lesions (Giasson, et al. (2000) Science 290:985-989), α-Syn inclusions in mice contained 3-nitro-tyrosine, were impregnated with silver and were stained with thioflavin S. 3-Nitro-tyrosine was only detected in a small number of inclusions, indicating that the modification was not required for the formation of inclusions in Tg mice.

An evaluation of the accumulation of insoluble and aggregated α-Syn in A53T α-Syn Tg mice was performed. The spinal cord, cerebellum and cortex of non-Tg (nTg) and Tg mice expressing wild-type (M7) and A53T (M83) human α-Syn were sequentially extracted with buffers with increasing strength of protein solubilization. Western blotting was used in conjunction with antibodies specific for mouse and human α-Syn (SNL-1) or only human α-Syn (LB509), as well as an anti-α-Syn antibody that preferentially recognizes pathological α-Syn (Syn 303) to detect α-Syn in these fractions. In nTg mice, the majority of α-Syn was extracted in the high-salt (HS) and HS/Triton X-100 fractions, but a small amount could also be observed in the RIPA fraction of the cortex. In M7 mice, the distribution profile was similar to nTg mice, but there was an increase in the o-Syn in the RIPA fraction. In M7 mice, the distribution profile was similar to nTg mice, but there was an increase in α-Syn in the RIPA fraction of all three anatomical regions analyzed, and there was also a small amount in the SDS-soluble fraction of the spinal cord and cerebellum. It is possible that this α-Syn in the SDS-fractions may correspond to protein aggregates that are not abundant enough to be seen by light microscopy. There was a significant accumulation of RIPA-insoluble (i.e. SDS- or formic acid (FA)-soluble) α-Syn in the spinal cord and cerebellum of M83 mice, and a lesser amount was also present in the cortex. RIPA-insoluble α-Syn in M83 mice was comprised of human A53T mutant α-Syn. Aggregated α-Syn that did not enter the resolving gels was also detected in the HS- and SDS-soluble fractions of the spinal cord of M83 mice. Furthermore, protein bands that are likely cross-linked dimers and trimers of α-Syn were detected in the SDS- and FA-soluble fractions of the spinal cord of M83 mice using antibody Syn 303. Aggregation of α-Syn in neuronal processes was also a major feature of Tg mouse line M91.

Ultrastructural analysis of pathological changes was conducted by toluidine blue staining semi-thin sections from the ventral root of nTg, M7 Tg mice and M83 Tg mice. Significant axonal degeneration and an increase in the endoneurial compartment in aged mice expressing A53T α-Syn was revealed. At the ultrastructural level, many degenerating axons associated with deteriorating myelin were observed. However, the initial phase of the degenerative process appeared to involve axons, since many severely degenerating axons were still surrounded by relatively intact myelin sheath. It appeared that following axonal degeneration, myelin sheath loosened and unraveled, eventually forming multi-lamellar myelin debris. Occasionally, these structures were surrounded by macrophages that appeared to remove debris. Longitudinal and axial views of axons filled with vacuoles suggested local blockage of axonal transport. Similarly, disarrayed bundles of NFs were also observed. Eosin/hematoxylin staining of the gastrocnemius muscle revealed sparse neurogenic muscle atrophy in A53T mice with impaired movement, consistent with a rapidly progressive axonal neuropathy.

Immunoelectron microscopy of the spinal cord of A53T mice demonstrated the intense and specific labeling of α-Syn inclusions in axon. Detachment and retraction of the axolemma could be observed in proximity of α-Syn inclusions, indicative of axonal degeneration. Aggregation of α-Syn in the periphery of the axolemma was also noted in some axons, a profile seen in human carriers of the A53T mutation. Higher magnification of immunolabeled α-Syn lesions demonstrated that they were predominantly comprised of ˜10-16 nm fibrils. The specific labeling directly on the fibrils indicates that these polymers are comprised of α-Syn. Perikaryal α-Syn inclusions were also comprised of similar immunolabeled filaments. Furthermore, α-Syn labeled fibrils were ultrastructurally distinct from NFs. α-Syn immunolabeled fibrils were much shorter and irregular and they did not have the side-arms characteristic of NFs (Lee and Cleveland (1996) Annu. Rev. Neurosci. 19:187-217; Julien and Mushynski (1998) Prog. Nucleic Acid Res. Mol. Biol. 61:1-23). To further confirm that inclusions were comprised of filamentous α-Syn, α-Syn fibrils biochemically isolated using a method previously developed for pathological human brain (Spillantini, et al. (1998) Proc. Natl. Acad. Sci. U.S.A. 95:6469-6473). Filaments were clearly immunolabeled with antibodies to 1-Syn in preparations from the cerebellum, spinal cord and pons from A53T α-Syn Tg mice. Furthermore, isolated α-Syn filaments were not labeled with antibodies to NFs in double-labeling experiments.

The Tg mouse model of the invention has many similarities with human neuronal α-synucleinopathies, especially familial PD due to the A53T α-Syn mutation. Foremost, mouse prion promoter-driven expression of human A53T α-Syn results in a mid-to-late onset neurodegenerative disorder that coincides with the accumulation of filamentous α-Syn cytoplasmic inclusions throughout the neuraxis, similar to patients with the A53T mutation. No α-Syn pathology was detected in young animals (<6 months) expressing A53T human α-Syn, and these animals did not present an overt neurological impairment. The onset of this disease is heralded by the appearance of sparse pathology without clinical manifestation, similar to humans (Formo (1996) J. Neuropathol. Exp. Neurol. 55:259-272). In striking contrast to Tg mice expressing A53T α-Syn, mice expressing wild-type human α-Syn did not develop any form of aberrant α-Syn accumulations or neurological defects.

Mice are often used for transgenic animal models because they are easy to house, relatively inexpensive, and easy to breed. However, other transgenic animals may also be made in accordance with the present invention such as, but not limited to, monkeys, bovine, sheep, rabbits and rats. Accordingly, a prion promoter of the invention may be derived from prions isolated from human, mouse, rat, sheep, or bovine.

It will be appreciated by those of skill in the art that animals expressing A53T α-Syn protein according to the present invention are useful for analysis of neurodegenerative diseases involving α-Syn pathologies. Such diseases involving α-Syn pathology, collectively referred to as α-synucleinpathies, include, but are not limited to, Alzheimer's disease (AD), Parkinson's disease (PD), Lewy body variant of Alzheimer's disease (LBVAD), dementia with Lewy body (DLB), neurodegeneration with brain iron accumulation type-1 (NBIA-1) (formerly known as Hallervorden-Spatz disease, and multiple system atrophy (MSA).

Signs or Symptoms of Neurodegenerative

Animals expressing A53T α-Syn protein are useful for the discovery and development of diagnostics and therapeutic compounds for treatment of neurodegenerative diseases, as well as for the better elucidation of the pathogenic mechanisms of these diseases.

In the development of new diagnostics, for example, an animal expressing A53T Cα-Syn may be used in assays of α-Syn inclusion detection or tests for neuronal injury in response to α-Syn inclusion formation.

Animals expressing A53T α-Syn protein may also be used to screen potential pharmaceutical compounds for their efficacy in preventing or delaying the development of any of neurodegenerative disease-related phenotypes seen in these animals. There is thus provided a method for screening candidate compounds for their efficacy in preventing or delaying the development of neurodegenerative diseases. The screening method comprises administering a candidate compound to an animals expressing A53T α-Syn protein prior to the appearance of a selected neurodegenerative disease-related phenotypic trait, and comparing the age at which the selected phenotypic trait appears in the treated animal to the age of appearance of that trait in an untreated animal. Neurodegenerative disease-related signs or symptoms of the disease which may be evaluated include, but are not limited to, the appearance of abnormal brain histology such as α-Syn inclusion formation or appearance of behavioral changes such as bradykinesia, rigidity, resting tremor, postural instability, periods of freezing or other motor impairments that may be determined, for example, by examining the performance of the animal in a rotarod task, as described herein.

Animals expressing A53T α-Syn protein may also be used to screen potential pharmaceutical compounds for their efficiency in ameliorating the symptoms of neurodegenerative diseases by similarly administering and comparing the effects of candidate compounds in animals after appearance of a selected neurodegenerative disease-related trait, such as abnormal brain histology or motor impairment.

Animals of the present invention which in a short time rapidly accumulate α-Syn inclusions in the brain may now be made and studied and used as a model to study possible therapies including pharmaceutical intervention, gene targeting techniques, antisense therapies, antibody therapies, etc. Furthermore, A53T α-Syn, in vitro cell lines may also be established and used in order to elucidate intracellular signaling systems involved in the disease as well as test and identify potentially therapeutic compounds.

Furthermore, the animals expressing A53T α-Syn protein of the invention may be used to examine situations or environmental hazards which are suspected of accelerating or initiating neurodegenerative diseases, such as for example, head trauma or toxic environmental agents. In this case, the animal may be exposed to a particular situation and then observed to determine motor impairment, premature death, etc. as indicators of the capacity of the situation to further provoke and/or enhance neurodegenerative diseases.

The animals of the present invention are useful for the more detailed characterization of neurodegenerative diseases to lead to elucidation of the pathogenesis of the progressive neurologic pathology and determination of the sequence of molecular events. The animals are useful for studying various proposed mechanisms of the pathogenesis of these diseases in order to lead to better treatments for the diseases.

Animals expressing A53T α-Syn protein of the invention are also useful for the identification of previously unrecognized genes which may also play a role in neurodegenerative diseases, either beneficial or deleterious. A transgenic animal bearing a candidate gene is crossed with an animals expressing A53T α-Syn protein of the invention and the effect of the presence of the candidate gene on the neurodegenerative disease-related traits of the transgenic animal are examined.

A candidate gene will be scored as beneficial if it delays or dilutes neurodegenerative disease-related phenotypes such as α-Syn inclusion formation and motor impairment.

Conversely, a candidate gene will be scored as favoring the development of neurodegenerative diseases if it advances the age of onset or enhances the penetrance of neurodegenerative disease-related phenotypes such as α-Syn inclusion formation and motor impairment.

The invention is further illustrated by the following, non-limiting examples.

### EXAMPLE 1

Generation of α-Syn Tg Mice

Wild-type human α-Syn cDNA (Jakes, et al. (1994) FEBS Lett. 345:27-32) and the same cDNA harboring the A53T mutation were cloned into the MoPrP.Xho expression vector (Borchelt, et al. (1996) Genet. Anal. 13:159-163) at the XhoI restriction site. The 14-kb NotI linear fragments containing the α-Syn cDNA and the mouse prion protein (PrP) gene promoter together with its 5′ untranslated region (UTR) containing an intron and its 3′ UTR sequences were used as the transgene to create α-Syn Tg mice in a C57Bl/C3H background. The Tg DNA was micro-injected into C57Bl/C3H mouse eggs. Genomic DNA samples were isolated from mouse tails with the Puregene DNA isolation kit in accordance with the manufacturer's instructions (Gentra Systems, Minneapolis, Minn.). Potential founders were identified by Southern blot analysis with a ³²P-labeled oligonucleotide-primed α-Syn DNA probe. Stable Tg lines carrying the wild-type (lines M7, M12 and M20) or mutant A53T (lines M83 and M91) human α-Syn constructs were established, and Tg and wild-type offspring were identified by Southern blot analysis of tail DNA. Homozygous Tg lineages were identified by quantitative Southern blot analysis and verified by backcrossing.

### EXAMPLE 2

Rotarod Task

Locomotor function was evaluated using a Rota-Rod treadmill Model 7650 (Ugo Basile, Comerio, Italy) set with accelerating revolution (4 to 40 revolution per minute) over a five-minute period. Mice were given 3 trials a day for 3 consecutive days.

### EXAMPLE 3

Antibodies

SNL-1 and SNL-4 rabbit antibodies were raised to synthetic peptides corresponding to amino acids 2-12 and 104-119 in α-Syn, respectively (Giasson, et al. (2000) J. Neurosci. Res. 59:528-533). Syn 211, Syn 204, Syn 208 and LB 509 correspond to mouse monoclonal antibodies specific for human α-Syn, while Syn 102 and Syn 202 correspond to mouse monoclonal antibodies that bind both α- and β-Syn (Giasson, et al. (2000) J. Neurosci. Res. 59:528-533). Syn 207 is a β-Syn-specific mouse monoclonal antibody (Giasson, et al. (2000) J. Neurosci. Res. 59:528-533) and gamma-1 is a γ-Syn-specific rabbit antibody (Giasson, et al. (2001) Exp. Neurol. 172:354-362). Syn 303, Syn 505, Syn 506, and Syn 514 are mouse monoclonal antibodies raised to oxidized human α-synuclein (Giasson, et al. (2000) Science 290:985-989). Although these antibodies do not recognize a specific oxidation modification, they preferentially recognize pathological α-Syn inclusions. nsyn 823 is a nitration-specific antibody raised to nitrated α-Syn (Giasson, et al. (2000) Science 290:985-989). Murine monoclonal antibodies RMO32 and RMO55 are specific for phosphorylated NF mid-size subunit (NFM), and RMO24 is specific for phosphorylated NF heavy subunit (NFH) (Schmidt, et al. (1991) Am. J. Pathol. 139:53-65). Antibody 17026 is tau-specific rabbit polyclonal antibody and PHF-1 is mouse monoclonal antibody specific for a phosphorylation epitope in tau. Anti-NSE (Polysciences, Inc., Warrington, Pa.), anti-GFAP (DAKO, Glostrio, Denmark) and anti-TH (Pelfreeze, Rogers, Ark.) are rabbit polyclonal antibodies. Anti-ubiquitin (MAB1510) was purchased from Chemicon International, Inc. (Temecula, Calif.).

### EXAMPLE 4

Gel Electrophoresis and Western Blotting

Mouse tissues were dissected and disrupted in 2% SDS, 50 mM Tris, pH 6.8 by sonication and heating to 100° C. for 10 min. Protein concentrations were determined using the biocinchoninic acid (BCA) assay (Pierce, Rockford, Ill.). Western blot analysis was performed as previously described (Giasson, et al. (1999) J. Biol. Chem. 274:7619-7622). Quantitative Western blotting was performed using 125I-labeled protein A (NEN) as secondary antibody. NSE was included as an internal standard to monitor loading errors. The membranes were dried and exposed to a PhosphorImager plate. The radioactive signal was quantified using ImageQuant software (Molecular Dynamics, Inc., Sunnyvale, Calif.).

### Example 5

Immunocytochemistry

Mice, anesthetized with an intra-peritoneal overdose injection of xylazine and ketamine, were perfused with phosphate-buffered saline (PBS) followed by 70% ethanol/150 mM NaCl or PBS-buffered formalin. Following surgical removal of brain and spinal cord, tissue was fixed for another 24 h in the respective fixatives. Samples were dehydrated through a series of graded ethanol solutions to xylene at room temperature, infiltrated with paraffin at 60° C. as is well known in the art, for example Trojanowski, et al. ((1989) J. Comp Neurol. 310:365-376), and cut into serial 6 μm sections. Immunocytochemistry was conducted according to standard, well-known methods, for example (Duda, et al. (2000) J. Neuropathol. Exp. Neurol. 59:830-841).

### Example 6

Double-Labeling Immunofluorescence

Double-labeling immunofluorescence studies were performed by incubating sections with Syn 505 and anti-TH antibodies. Following extensive washes, sections were labeled using Alexa Fluor 488 (green) and 594 (red) conjugated secondary antibodies (Molecular Probes, Eugene, Oregon) and covered with Vectashield-DAPI mounting medium (Vector Laboratories, Burlingame, Calif.).

### Example 7

Spinal Cord Neuronal Counting

The L2-L3 spinal cord segments of Tg and nTg mice were formalin fixed and paraffin embedded. The entire spinal cord segments were cut in 12 μM thick sections, which were stained with cresyl violet. Motor neurons in the Rexed's laminae IX of the L2 and L3 spinal levels were counted by two observers.

### Example 8

Sequential Biochemical Fractionation

Cortex, cerebellum and spinal cord from 9 month-old mice were dissected, weighed and homogenized in 3 ml/g of HS buffer (50 mM Tris, pH 7.5, 750 mM NaCl, 5 mM EDTA, and a cocktail of protease inhibitors. The samples were sedimented at 100,000×g for 20 minutes. Pellets was re-extracted with HS buffer followed by two sequential extractions with 3 ml/g of HS buffer containing 1% Triton X-100 (HS/T fraction). The pellets were homogenized in 500 μl of HS buffer/1 M sucrose and after centrifugation the floating myelin was discarded. The pellets were extracted with 2 ml/g of RIPA (50 mM Tris, pH 8.0, 150 mM NaCl, 5 mM EDTA, 1% NP40, 0.5% sodium deoxycholate, 0.1% SDS) and sedimented at 100,000×g for 20 minutes. Half of each pelleted sample was extracted with 1 ml/g SDS-sample buffer (SDS fraction) by sonication and heating to 100° C. for 10 minutes or 70% formic acids (FA fraction) by sonication. FA was removed by lyophilization and the dried material was resuspended in 1 ml/g of SDS-sample buffer by heating to 100° C. for 10 minutes. Five μl of each fraction was loaded on separate lanes of 12% polyacrylamide gels, and the distribution of α-Syn was determined by western blotting analysis.

### Example 9

Conventional and Immunoelectron Microscopy

For direct electron microscopy, mice were deeply anesthetized and sacrificed by intracardiac perfusion with 0.1 M cacodylate buffer, pH 7.4 followed by 4% paraformaldehyde/2% glutaraldehyde in 0.1 M cacodylate, pH 7.4. The L5 segments of the spinal cord and L5 ventral roots were removed and further fixed for 18 hours. Tissue was post-fixed with 2% osmium tetraoxide for 1 hour, dehydrated with graded ethanol solutions, and embedded in Epon.

Mice prepared for pre-embedding immunoelectron microscopy were perfused with 0.1 M cacodylate buffer, pH 7.4 followed by 2% paraformaldehyde/0.5% glutaraldehyde in 0.1 M cacodylate, pH 7.4. The tissue was further fixed for 12 hours, washed with PBS, cut into 50 μM sections and reacted with 0.1% sodium borohydrate in PBS for 10 minutes. Following extensive, sections were labeled then labeled with antibody Syn 303 and sequentially incubated with a biotinylated goat anti-mouse antibody and ABC reagents. Following the reaction with DAB, tissue sections were further developed with silver methenamine as has been described, for example Rodriguez, et al. ((1984) Histochemistry 81:253-263). Sections were post-fixed with 1.5% glutaraldehyde and 1% osmium tetraoxide, and following dehydration in graded ethanol they were embedded in Epon.

### Example 10

Isolation of Dispersed α-Syn Filament

α-Syn filaments were extracted from the pons, cerebellum or spinal cord of 9 month-old motor impaired M83 mice using a well-known method for human tissue (Spillantini, et al. (1998) Proc. Natl. Acad. Sci. U.S.A. 95: 6469-6473). Briefly, tissue was homogenized in 50 mM Tris, pH 7.4, 750 mM NaCl, 2 mM EGTA, 10% sucrose. After centrifugation at 14,000×g for 20 minute, the supernatant was incubated with 1% sarcosyl for 30 minute. The solution was centrifuged at 100,000×g for 1 h and the resulting pellet was resuspended in 50 mM Tris, pH 7.4. Aliquots of the dispersed filaments were applied onto carbon-coated 300-mesh copper grids. Grids were blocked with 1% bovine serum albumin in PBS and filaments were immunolabeled with anti-α-Syn antibodies followed by a goat anti-mouse antibody secondary conjugated to 10 nm gold. Fibrils were visualized by negative staining with 1% uranyl acetate.

What is claimed is:

1. A transgenic animal comprising a nucleotide sequence encoding a heterologous α-synuclein protein operably linked to at least a portion of a regulatory region of a prion gene, wherein said transgenic animal expresses human α-synuclein protein.

2. The transgenic animal of claim 1 wherein the animal displays an accelerated appearance of α-synucleinopathies.

3. A method of screening for a drug for treatment of a neurodegenerative disease comprising administering a drug to the transgenic animal of claim 1 and determining if the drug decreases at least one of the signs or symptoms of a neurodegenerative disease.

4. A method of screening for a drug that decreases formation of α-synuclein inclusions comprising administering a drug to the transgenic animal of claim 1 and determining if the drug decreases formation of α-synuclein inclusions in the transgenic mouse.
